SEQUENCE LISTING (6 sequences)

SEQ ID NO: 1 - DNA, 3918 bp, Homo sapiens
aaacagagtgtggtcaggggcagttttggcaataaggagtgtgtgtgcgtgtgtgtatctctcggggtgccaagtgagaccctatttcccagcactaaggagggtggttcgggtgatcatctcagagggctgcattttggggtgttgggaggggctggaattgaggcagtaactctgggtgcgtcccggcgctgggggtaagaccctgagttcagggatgtggcggcagcaacctggctgtgctgagatggggacaggatgaaactgagtggtaagtgacaggattgtatgtcttagggtgtgaggctgtgccagtgtgaccctacttctcagggcaggaggcgagtctttgtgtcttaggacgtgtgtcacaggtgtcactcagcccatcttaaaggagccagtcactggggagagtccccaggtcgccaggcctcctggttcccctgccccctttcagcctcctcccaggatatgactcgctgtggccctagccggggcttaagcccctgtataaataggcggatcccctggccaggctgggcctgacccaagctggtctacccgaggccctgcccaccagcacccaccccgacatccacgaaggctttggcagggcagggggggcattgctggccccacagaattggagttagcctctctagctccaaagccccagggaggggggcagtggcaccccctgcagctgccccagcccgccccagacgacgctcacattttccagtgtttagattttatccgctttattaatgaggcaagaggcccgatcctgggggggaagggggctgctcccgccctgctgggaggagggggtcacatggggccagaccaactccagggagcctcactcctcgtggagggagccgctggggcccagagtggcccccgcccattgccaggagcagagaggggggtctctgccactccaggcccccagcgggcgtgcgcggggtggagcggcccctggaggccagcaagggcggcgggaggagctgaggagccgccccaggaagagggagggaggaagcggcttgccggagagccagggcgcagtgggcggcagggctgagcggccggtgatggggaccccacatcccaggcagtgccgggtaagcgggttgcacatgagggtccctgattggggaggaggagggcatgaaggggtggggccatggccacctgctgtccgtcctgtgtccccgggctcttctccctgatctgtgcgtccctgtcttaccgtctgtccatcctcccttcccatctggcctggtgggcagttccgccctttcagccctcaccaccaggatatcaaagagtggatggtcgtggggccgcctccaggcctctggggcagggatatggagtggaaccgtgggcacccagggtgaggcagaggtgatcggggtagggcctggcgagagaccgcagtggagcaggggcaggtccatggggcaggggcagagatgggtccatggcacagatgtggctgtgccatgtggggtcagagatgggtccatgggggacagggacatggccgtggggagagatgtgcccgtggtggtagagatggggctggggcatcgaggatgggactggagtaaagctgacatggggaaagtcaggtagggccacagtggcagaaatgggtgagagctatcagatggagccacaggccccaggaatttgctgggtgtaaaaatggaaggtgggggtcggaggcactggcagagatgcctgagggcggggctggggggaatcttgcaggaaaccatgaagggcagagaaagggccagtggggttagagggaggccctggagcaggagatggggtggtgagaggcaaaagagagggagaagggtttccaaatggagtggccaggtcatttggagttgcccatggcaactgccatgggcagaggggccgcctgagaacgccatggagtcaaacaggccctgatgttctgagatggcaccgtgggctggtccccgcccgggcccagccagcctcactctgccctcctctcctctatccagcacccctggcgcctgacatgagcccttgcgggcccctcaacctgagcctggcgggcgaggcgaccacatgcgcggcgccctgggtccccaacacgtcggccgtgccgccgtcgggcgcttcgcccgcgctgcccatcttctccatgacgctgggcgccgtgtccaacctgctggcgctggcgctgctggcgcaggccgcgggccgcctgcgacgccgccgctcggccgccaccttcctgctgttcgtggccagcctgctggccaccgacctggcgggccacgtgatcccgggcgcgctggtgctgcgtctgtacactgcggggcgcgctccggccggcggggcctgccacttcctgggcggctgcatggtcttcttcggcctgtgcccgctgctgctgggctgtggcatggccgtggagcgctgcgtgggcgtcacgcggccgctgctccacgccgcgcgggtctcggtcgcccgcgcgcgcctggcgctggccgcggtggccgcggtggccttggccgtggcgctgctgccgctggcgcgcgtgggccgctatgagctgcagtacccgggcacgtggtgcttcatcggcctgggtcccccgggcggctggcgccaggcactgcttgctggcctcttcgccagcctcggcctggtcgcgctcctcgccgcgctggtgtgcaacacgctcagcggcctggccctgctacgcgcccgctggcgacgccgctcccgacggcctcccccggcctcaggccccgacagccggcgtcgctggggggcgcacggaccccgctcggcctccgcctcgtccgcctcgtccatcgcttcggcctccaccttctttggcggctctcggagcagcggctcggcacgcagagctcgcgcccacgacgtggagatggtgggccagcttgtcggtatcatggtggtgtcgtgcatctgctggagcccaatgctggtgaggggcgcaccggcccctcgagccacgctccttcccgctccctctcggcaccctcccgccctttgtcgtcccaggacacctggggcctccatcctggactcaaccaaggccccggcccctagaggccccacctgccccgaaagccagcatcgccttctccatctgacctcccatccttcctcctagcccccctctcctcttcctttttggggtctttgtagcgcaccccgacccacacaagcctcctctcctgccccaccgttataagtcgccgcgcttcattccctagtcctttcacccaacccccttgcttttcctctttccgggacacctgagactcctctacggccggacccccacccactgaaggtgtttgtttctttgccccccttttttttccgcatccgtttctcatctggatccccatttaccctccctgcgacggctcgcccctcctcccaggcttttaccttccagccacgcccccaccatccctgcgcccccctgcgcccgcgcctgctctatagctcacaccggcttcccccgcaggtgttggtggcgctggccgtcggcggctggagctctacctccctgcagcggccactgttcctggccgtgcgccttgcctcctggaaccagatcctggacccttgggtgtacatcctactgcgccaggccgtgctgcgccaactgcttcgcctcttgcccccgagggccggagccaagggcggccccgcggggctgggcctaacaccgagcgcctgggaggccagctcgctgcgcagctcccggcacagcggcctcagccacttctaagcacaaccagaggcccaacgactaagccagcccaccctgggctgggcccaggtgcgcggcgcagagcctttgggaacttgggccag

SEQ ID NO: 2 - DNA, 22 bp, Artificial Sequence
  Description of Artificial Sequence Primer
cttcatgccctcctcctcccta

SEQ ID NO: 3 - DNA, 19 bp, Artificial Sequence
  Description of Artificial Sequence Primer
cctgccccatggacgggcc

SEQ ID NO: 4 - DNA, 26 bp, Artificial Sequence
  Description of Artificial Sequence Primer
tctgatagctctcacccattttggcc

SEQ ID NO: 5 - DNA, 24 bp, Artificial Sequence
  Description of Artificial Sequence Primer
tccacggccatgccacagccccgc

SEQ ID NO: 6 - DNA, 24 bp, Artificial Sequence
  Description of Artificial Sequence Primer
ggaggcaaggcgcacggccaggac

(19) United States
(12) Patent Application Publication
(10) Pub. No.: US 2002/0076702 A1
(43) Pub. Date: Jun. 20, 2002

(54) Methods

(76) Inventors: Rakesh Anand, Macclesfield (GB); John C. Smith, Macclesfield (GB); John E. N. Morten, Macclesfield (GB)

Correspondence Address: Pillsbury Madison & Sutro LLP, Intellectual Property Group, East Tower, Ninth Floor, 1100 New York Avenue, N.W., Washington, DC, 20005-3918, US

(21) Appl. No.: 09/781,311

(22) Filed: Feb. 13, 2001

(30) Foreign Application Priority Data
Feb. 17, 2000 (GB) 0003553.5
Apr. 6, 2000 (GB) 0008376.6

(51) Int. Cl. C12Q 1/68; C07H 21/04

(52) U.S. Cl. 435/6; 536/24.3

(57) ABSTRACT

This invention relates to polymorphisms in the human prostaglandin E2 receptor 1 (EP1-R) gene and corresponding novel allelic polypeptides encoded thereby. Fourteen specific polymorphisms are identified. The invention also relates to methods and materials for analysing allelic variation in the EP1-R gene and to the use of said polymorphism in the diagnosis and treatment of EP1-R ligand mediated diseases, such as cancer or arthritis.

### FIELD OF THE INVENTION

The present invention relates to allelic polymorphisms of the genes encoding human prostaglandin receptors, in particular the prostaglandin receptor families designated FP and EP1. The present invention further relates to the detection of these polymorphisms in a subject for whom such information is useful for treatment, diagnostic and/or prognostic purposes.

### BACKGROUND OF THE INVENTION

Prostaglandin Receptors

The prostaglandin receptor family encompasses at least five classes of receptors, designated FP, EP, IP, DP, and TP receptors, which are classified based on their sensitivity to the five primary prostanoids (F2α, E₂, I₂, D₂, and TXA₂). EP receptors further comprise four subtypes, designated EP1-4, which differ in their responses to various agonists and antagonists. Furthermore, ligand binding studies have shown a certain degree of cross-reactivity between receptors (Coleman et al., Pharm. Rev., 46:205-229, 1994).

Each of the above-identified receptors possesses seven hydrophobic transmembrane domains, which are characteristic of the rhodopsin-type receptor superfamily. The high degree of structural homology between the different receptors also suggests that they may derive from a common ancestral gene. The genes for all the receptors are apparently formed from three exons, wherein the first exon contains 5′-untranslated sequences; the second exon contains the majority of the protein-coding sequence; and the third exon contains the carboxyterminal end of the protein-coding sequence (from the sixth transmembrane domain and downstream) and 3′-untranslated sequences.

These seven-transmembrane-domain receptors display several important structural/functional domains, including, for example, (i) the three extracellular loops which form the prostanoid-binding site and (ii) the intracellular domains, preferably the third, and possibly also parts of the intracellularly located carboxyterminal domain, which interact with a G-protein to initiate a signal transduction pathway. Furthermore, a conserved arginine residue (at position 60) (located in the seventh transmembrane domain) may bind to the α-carboxylic acid of prostanoid ligands. Consistent with this idea, individuals carrying a mutation that results in a substitution of Leu for this Arg residue exhibit impaired platelet aggregation (Ushikubi, et al., Throms. Haemostst, 57:158 (1987); Hirata, et al., Nature 349:617 (1994); Fuse, et al., Blood; 81:994(1993)).

Role of Prostaglandins in Physiology

Role of Prostaglandins in the Cardiovascular System

The prostanoids are known to act in multiple ways in the human pulmonary vascular system (Jones et al., Clin. Exp. Pharmacol. Physio. 24:969-72, 1997). Four type of prostanoid receptors are present on pulmonary arterial vessels in humans: thromboxane (TP) receptors mediate constriction and are blocked by antagonists, such as BAY u 3405, GR 32,191, and EP 169; prostaglandin (PG) E.P.₃ receptors also mediate constriction, and are agonized by the compounds S C 46,275, solprostone, misoprosto, and prostaglandin E2 (PGE₂). PGE₂ causes relaxation in a few pulmonary artery preparations, and an EP₂ may be involved (Jones et al., supra). Prostacyclin produces relaxation, possibly by potassium channel opening (Jones et al., supra). In addition to the prostanoids discussed above, losartin, a non-peptide angiotensin to antagonists, interacts with thromboxane A2/prostaglandin H2 receptors, and inhibits prostanoid-induced beta constriction in canine coronary arteries and platelet application and vaso constriction in hypertensive rats (Li et al., J. Cardiovasc. Pharmacol. 32:198-205, 1998). Previously studies have shown that prostanoids play a role in rennin-dependent and rennin-independent hypertension (Lin et al., Hypertension 17:517-25, 1991), prostaglandins have also been reported to be involved in the development in clinical expression of arteriosclerosis (Hirsh et al., M. J. Med. 71:1009-26, 1981).

Role of Prostaglandins in the Pulmonary System

Prostaglandins have also been reported to play an important role in pulmonary hypertension and pulmonary health. Prostaglandin synthesis inhibitors administered in utero are associated with pulmonary hypertension of the fetus and, in the case of humans, children (Wendelberger, Semin. Perinatol. 11:1-11, 1987). Prostaglandin receptors have been localized to lung tissue and appear to play a role in pulmonary development and function.

Glaucoma and Intraocular Pressure

Patients suffering from glaucoma exhibit an increased intraocular pressure (IOP). This condition is not only painful, but can also, when left untreated, lead to permanent damage to the blood vessels in the eye. Interference with blood flow to ocular tissues over time further leads to a serious impairment of vision.

Among the drugs that are currently used to treat IOP are synthetic prostaglandin analogues. These compounds bind to prostaglandin receptors in the eye and thereby reduce IOP by activating a G-protein coupled pathway.

The prostaglandin derivatives bind with varying degrees of specificity and selectivity to different prostaglandin receptors, which can lead to complex physiological responses in the patient being treated. In addition, different prostaglandins may be vasoconstrictors or vasodilators; may contract or relax smooth muscle (including bronchial, tracheal or uterine muscles); and may affect platelet function, immune cell chemotaxis, B-cell differentiation, and other aspects of immune system physiology, as well as kidney function and endocrine and metabolic processes.

The high incidence of hypertension and glaucoma, and the serious clinical consequences of these conditions, mean that there is a need for methods and compositions that allow the identification of the therapeutic regimen that will result in a more positive treatment outcome. It would also be useful to be able to identify individuals who are at risk for toxic or abnormal responses to prostanoid treatment. Accordingly, there is a need in the art to identify and characterize genetic markers, i.e., patterns of allelic polymorphisms, within prostaglandin receptor genes that are associated with positive treatment outcomes utilizing specific therapeutic regimens.

### SUMMARY OF THE INVENTION

The invention advantageously provides a method for assessing prostanoid response status in an individual to be tested. The method comprises comparing two polymorphic patterns. The first pattern is a test polymorphic pattern comprising at least one polymorphic position within a prostaglandin receptor gene of the individual. The second is a reference polymorphic pattern derived from a population of individuals exhibiting a predetermined prostanoid response status. The comparison of the test polymorphic pattern with the reference polymorphic pattern provides for concluding whether the individual possesses the prostanoid response status. Such a conclusion is based on whether the test pattern matches the response pattern.

The present invention provides isolated nucleic acids encoding polymorphic variants of human FP and EP-1 prostaglandin receptors. In the FP receptor, non-limiting examples of polymorphisms include changes at one or more of nucleotides 63, 213, 465, 573, and 1012 of the nucleic acid sequence depicted in FIG. 1. In the EP-1 receptor, non-limiting examples of polymorphisms include changes at one or more of nucleotides 211, 264, 689, 690, 767, 816, and 999 of the nucleic acid sequence depicted in FIG. 2. The invention also provides recombinant DNA vectors comprising these nucleic acids, cells comprising the vectors, and methods for producing variant FP and EP-1 polypeptides that are carried out by culturing the cells under conditions that permit expression of FP or EP-1 polypeptides.

In another aspect, the invention provides methods for detecting FP or EP-1 receptor polymorphisms in a human subject, which are carried out by the steps of: (i) obtaining a DNA sample from the subject; (ii) individually amplifying the DNA regions containing FP or EP-1 genes; and (iii) determining the presence or absence of one or more polymorphisms within the amplified DNA. Such polymorphisms are optionally linked with one or more other polymorphisms in a polymorphism pattern, which is correlated with a predisposition to disease or disorder, or with responsiveness to a given treatment.

The pattern of FP and/or EP-1 allelic polymorphisms, either alone or in combination with the allelic patterns of other genes, can be used.

### BRIEF DESCRIPTION OF THE DRAWINGS

FIG. 1. Illustration of the nucleotide (SEQ ID NO:1) and amino acid (SEQ ID NO:2) sequence of the human FP prostaglandin receptor, including allelic polymorphisms. Nucleotide positions carrying genetic variations are underlined and highlighted. The genetic variations are indicated above these sites, designated R (G->A) or Y (C->T). Amino acid residues that are changed are underlined.

FIG. 2. Illustration of the nucleotide (SEQ ID NO:1) and amino acid (SEQ ID NO:2) sequence of the human EP-1 prostaglandin receptor protein-coding sequence, including allelic polymorphisms. Nucleotide positions carrying genetic variations are underlined and highlighted. The genetic variations are indicated above these sites, designated R (G->A) or Y (C->T). Amino acid residues that are changed are underlined.

FIG. 3. Schematic illustration of the predicted intramembrane topology of the human FP and EP1 prostaglandin receptors. The positions of the polymorphisms identified by the present invention are indicated.

### DETAILED DESCRIPTION OF THE INVENTION

The present invention is based, in part, on the observation that genetic variation in these receptors may correlate with changes in function and that certain patterns of these genetic variations, which are termed herein polymorphism patterns, can be used to predict predisposition to develop a disease or responsiveness to a particular treatment By way of explanation, without being limiting, sequence alterations in the extracellular domains may influence the specificity and selectivity of ligand binding. Similarly, sequence alterations in the G-protein-interacting region could disrupt or enhance G-protein interaction, or result in binding to a different G-protein than normally occurs. These and other genetic variations are thought to result in a variety of different response patterns to a particular prostanoid drug. For example, certain individuals within the general population, as a result of the particular sequence of their prostaglandin receptors, may be “super-responders” to particular prostanoid drugs, or conversely, may lack the ability to respond. Still other individuals may suffer toxic effects from a particular drug if the drug interacts with the wrong receptor, or if the receptor binds to the wrong G-protein. Thus, it is contemplated that the allelic pattern of prostaglandin receptors in an individual can be used to predict the nature of that individual's response to prostaglandin treatment.

The invention provides a powerful predictive tool for clinical testing of and treatment with prostanoids. For clinical testing, the present invention permits smaller, more efficient clinical trials by identifying individuals who are likely to respond poorly to a treatment regimen and reducing the amount of uninterpretable data. By evaluating a test individual's polymorphism pattern, a physician can prescribe a prophylactic or therapeutic regimen customized to that individual's prostanoid response status. Adverse responses to particular therapies can be avoided by excluding those individuals whose prostanoid response status puts them at risk for that therapy. Appropriate changes in lifestyle, including diet and environmental stress can be prescribed for individuals whose test polymorphic pattern matches a reference pattern that correlates with increased predisposition to develop a condition, such as glaucoma, that can be treated with prostanoids.

In practicing the present invention, the presence of different prostaglandin receptor, particularly FP and/or EP-1, alleles in an individual patient can be determined by either. 1) molecular detection of the DNA or RNA encoding FP and/or EP-1 variants using nucleic acid probes, with or without nucleic acid sequencing (“genotypic characterization”) or 2) immunological detection of the FP and/or EP-1 polypeptides (“phenotypic characterization”). Non-limiting examples of tissues expressing FP include corpus luteum, uterus (myometrium), and iris sphincter. Non-limiting examples of tissues expressing EP-1 include muscle, myometrium, kidney, lung, and iris sphincter. For example, ocular tissues or vascular tissues can be used for phenotypic characterization.

### Definitions

“Prostanoid response status” as used herein refers to the physiological status of an individual resulting from the function of the person's prostaglandin receptors. Prostanoid response status may be as reflected in one or more status markers or indicators including genotype. Prostanoid response status shall be deemed to include without limitation not only the absence or presence of a pathology or disease in one or more components of the individual's prostaglandin receptor function and the individual's predisposition to developing such a condition, but also the individual's responsivity, i.e., the ability or inability of the individual to respond (positively or negatively) to a particular prophylactic or therapeutic regimen or treatment with a prostanoid, particularly a prostaglandin. A negative response includes one or more adverse reactions and side effects. Status markers include without limitation clinical measurements such as, e.g., blood pressure, inflammation, heart rate, intraocular pressure, and other physiological responses mediated by prostaglandins, etc.

The term “prostanoids” as used herein refers to a compound that binds to a prostaglandin receptor. The term encompasses agonists and antagonists of prostaglandin receptor. In a specific embodiment, a prostaglandin is a prostanoid. Other prostanoids include, but are by no means limited to thromboxane agonists (e.g., BAY u-3405, GR 32,191, U46619, EP 169) and antagonists (e.g., SQ 29,548), EP3 receptor agonists (SC 46,275, sulprostone, misoprostol), prostacyclin and its agonists (BMY 45,778, the diphenylindole Cu23), ilsoprost, losartan (a non-peptide angiotensin-II antagonist), and the like.

The term “prostaglandin-associated syndrome” refers to a-disease or disorder that is mediated, at least in part, by prostaglandin or prostaglandin receptors. In particular embodiments, an increase or a decrease in prostaglandin activity is correlated with the condition. In other embodiments, an increased or decreased response of prostaglandin receptors is correlated with the condition.

Status markers according to the invention are assessed using conventional methods well known in the art. Also included in the evaluation of prostanoid response status are quantitative or qualitative changes in status markers with time, such as would be used, e.g. in the determination of an individual's response to a particular therapeutic regimen or of a predisposed individual's eventual development of a disease condition. One such condition is cardiovascular disease, and particularly hypertension. Another condition is pulmonary disease. Still another such condition is intraocular pressure resulting in glaucoma.

“Intraocular pressure” (IOP) as used herein refers to the pressure in the aqueous humor of the eye, which is routinely measured using, e.g., a Schiotz tonometer.

“Glaucoma” as used herein refers to an increase in IOP over 15-20 mM Hg, which may lead to optic nerve damage and consequent blindness.

Examples of prostaglandin-associated syndromes that are included in the foregoing definition of prostanoid response status include diagnosis of, or predisposition to, one or more syndromes, such as, e.g., cardiovascular disease, particularly hypertension, glaucoma, inflammatory diseases, etc. It will be understood that a diagnosis of a syndrome made by a medical practitioner encompasses not only clinical measurements but also medical judgment.

“Responsivity”, as used herein, refers to the type and degree of response an individual exhibits to a particular therapeutic regimen, i.e., the effect of a treatment with a prostanoid, including a prostaglandin, on an individual. Responsivity breaks down into three major categories: therapeutic effect; no effect; and adverse effect Naturally, there can be differing degrees of a therapeutic effect, e.g., between full elimination and partial elimination of symptomology. In addition, adverse effects, or side effects, may be observed even though the treatment is beneficial, Le., therapeutically effective. Indeed, the present invention may permit identification of individuals with complex responsivity traits or patterns.

A “predisposition to develop a prostaglandin-associated syndrome” refers to an increased likelihood, relative to the general population, to develop a prostaglandin-associated syndrome, as defined above. A predisposition does not signify certainty, and development of the syndrome may be forestalled or prevented by prophylaxis, e.g., adopting a modified diet, or treatment with gene therapy or pharmaceuticals. Naturally, an advantage of the present invention is that it permits identification of individuals who are, based on their genotype, predisposed to develop such a syndrome, and for whom prophylactic intervention can be especially important. In a preferred embodiment, the syndrome is hypertension. In another embodiment, the syndrome is glaucoma.

A “polymorphism” as used herein denotes a variation in the nucleotide sequence of a gene between individuals. Genes that have different nucleotide sequences as a result of a polymorphism are “alleles.” A “polymorphic position” is a predetermined nucleotide position within the sequence of the gene. In some cases, genetic polymorphisms are reflected by an amino acid sequence variation, and thus a polymorphic position can result in location of a polymorphism in the amino acid sequence at a predetermined position in the sequence of a polypeptide. An individual “homozygous” for a particular polymorphism is one in which both copies of the gene contain the same sequence at the polymorphic position. An individual “heterozygous” for a particular polymorphism is one in which the two copies of the gene contain different sequences at the polymorphic position.

A “polymorphism pattern” as used herein denotes a set of one or more polymorphisms, including without limitation single nucleotide polymorphisms, which may be contained in the sequence of a single gene or a plurality of genes. In the simplest case, a polymorphism pattern can consist of a single nucleotide polymorphism in only one position of one of two alleles of an individual (allelic polymorphism). One always has to look at both copies of a gene. A polymorphism pattern that is appropriate for assessing a particular aspect of prostanoid response status (e.g., predisposition to hypertension or glaucoma) need not contain the same number (nor identity, of course) of polymorphisms as a polymorphism pattern that would be appropriate for assessing another aspect of status (e.g., responsivity to ACE inhibitors or angiotensin-II antagonists for control of hypertension). A “test polymorphism pattern” as used herein is a polymorphism pattern determined for a human subject of undefined status. A “reference polymorphism pattern” as used herein is determined from a statistically significant correlation of patterns in a population of individuals with predetermined status.

“Nucleic acid” or “polynucleotide” as used herein refers to purine- and pyrimidine-containing polymers of any length, either polyribonucleotides or polydeoxyribonucleotides or mixed polyribo-polydeoxyribo nucleotides. Nucleic acids include without limitation single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases and non-naturally occurring phosphoester analog bonds, such as phosphorothioates and thioesters. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “oligonucleotide” refers to a nucleic acid, generally of at least 10, preferably at least 15, and more preferably at least 20 nucleotides, that is hybridizable to a genomic DNA molecule, a cDNA molecule, or an mRNA molecule encoding a gene, cDNA, mRNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a gene of interest, or to detect the presence of nucleic acids encoding the gene of interest. In a further embodiment, an oligonucleotide of the invention can form a triple helix with a double stranded sequence of interest in a DNA molecule. In still another embodiment, a library of oligonucleotides arranged on a solid support, such as a silicon wafer or chip, can be used to detect various polymorphisms of interest. Generally, oligonucleotides are prepared synthetically, preferably on a nucleic acid synthesizer. Accordingly, oligonucleotides can be prepared with non-naturally occurring phosphoester analog bonds, such as thioester bonds.

An “isolated” nucleic acid or polypeptide as used herein refers to a nucleic acid or polypeptide that is removed from its original environment (for example, its natural environment if it is naturally occurring). An isolated nucleic acid or polypeptide contains less than about 50%, preferably less than about 75%, and most preferably less than about 90%, of the cellular components with which it was originally associated.

A nucleic acid or polypeptide sequence that is “derived from” a designated sequence refers to a sequence that corresponds to a region of the designated sequence. For nucleic acid sequences, this encompasses sequences that are identical to or complementary to the sequence.

A “probe” refers to a nucleic acid or oligonucleotide that forms a hybrid structure with a sequence in a target nucleic acid due to complementarity of at least one sequence in the probe with a sequence in the target nucleic acid. Generally, a probe is labeled so it can be detected after hybridization.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989, Molecular Cloning: A Laboratory Manual, Second Edition, Cold Spring Harbor Laboratory Press: Cold Spring Harbor, N.Y.). The conditions of temperature and ionic strength determine the “stringency” of the hybridization. For preliminary screening for homologous nucleic acids, low stringency hybridization conditions, corresponding to a Tₘ of 55° C., can be used, e.g., 5× SSC, 0.1% SDS, 0.25% milk, and no formamide; or 30% formamide, 5× SSC, 0.5% SDS). Moderate stringency hybridization conditions correspond to a higher Tₘ, e.g., 40% formamide, with 5× or 6× SCC. High stringency hybridization conditions correspond to the highest Tₘ, e.g., 50% formamide, 5× or 6× SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA. DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

A “gene” for a particular protein as used herein refers to a contiguous nucleic acid sequence corresponding to a sequence present in a genome which comprises (i) a “coding region,” which comprises exons (i.e., sequences encoding a polypeptide sequence or “protein-coding sequences”), introns, and sequences at the junction between exons and introns; and (ii) regulatory sequences flanking the 5′ and 3′ ends of the coding region.

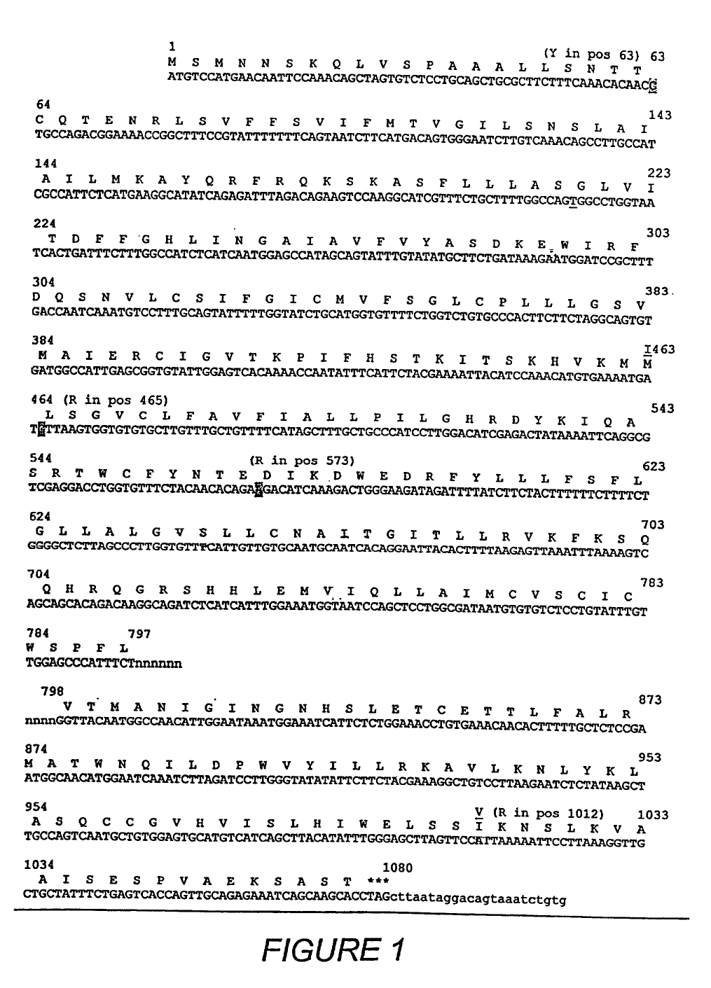

The “FP prostaglandin receptor” as used herein refers to a prostaglandin receptor having the amino acid sequence depicted in FIG. 1. The FP receptor is identified functionally as a receptor that binds the naturally occurring prostanoid F2a.

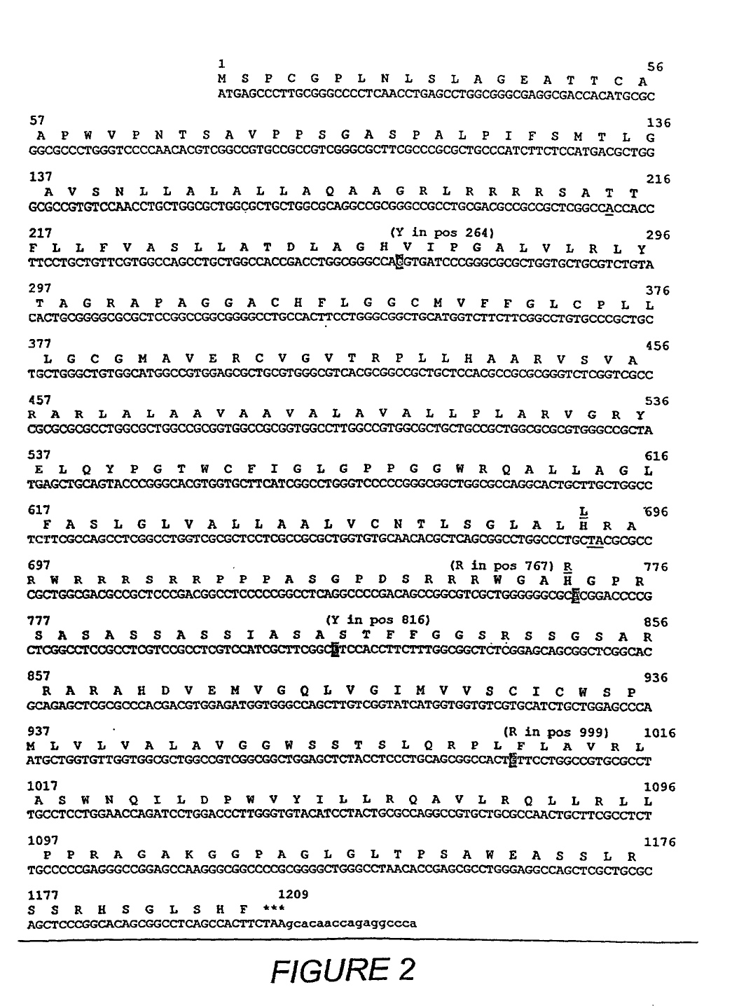

The “EP-1 prostaglandin receptor” as used herein refers to a prostaglandin receptor having the amino acid sequence depicted in FIG. 2. The EP-1 receptor is identified functionally as a receptor that binds the naturally occurring prostanoid E₂.

Methods for Assessing Prostanoid Responsivity Status

The present invention provides methods for assessing prostanoid response status in a human individual, i.e., for identification of allelic patterns in prostaglandin receptor genes, such as genes encoding FP and EP-1. The methods are carried out by comparing a polymorphic position or pattern (“test polymorphic pattern”) within the individual's gene encoding a prostaglandin receptor with the polymorphic patterns of humans exhibiting a predetermined prostanoid response status (“reference polymorphic pattern”). If the prostanoid responsivity status is the prediction of responsivity to a therapy, a single polymorphic position can provide a pattern for comparison. However, it is preferable to use more than one polymorphic position for the pattern to improve the accuracy of the prediction If the prostanoid responsivity status is predisposition to a prostaglandin-associated syndrome, preferably at least two, and more preferably at least three, polymorphic positions are used to establish the pattern.

For any meaningful prediction, the polymorphic pattern of the individual is identical to the polymorphic pattern of individuals who exhibit particular status markers, cardiovascular syndromes, and/or particular patterns of response to therapeutic interventions.

In one embodiment, the method involves comparing an individual's test polymorphic pattern with reference polymorphic patterns of individuals who have been shown to respond positively or negatively to a particular therapeutic regimen. Therapeutic regimen as used herein refers to treatments aimed at the elimination or amelioration of symptoms and events associated prostaglandin-associated disease. Such treatments include without limitation one or more of alteration in diet, lifestyle, and medication regimen. It is contemplated, for example, that patients who are candidates for a particular therapeutic regimen will be screened for polymorphic patterns that correlate with responsivity to that particular regimen.

In another embodiment, the method involves comparing an individual's polymorphic pattern with polymorphic patterns of individuals who exhibit or have exhibited one or more markers of prostaglandin-associated disease, such as, e.g., cardiovascular disease, such as hypertension, glaucoma, inflammatory diseases, etc. and drawing analogous conclusions as to the individual's responsivity to therapy, predisposition to developing a syndrome, etc, as detailed above.

Identification of Polymorphic Patterns

In practicing the methods of the invention, an individual's polymorphic pattern can be established, e.g., by obtaining DNA from the individual and determining the sequence at a predetermined polymorphic position or positions in a gene, or more than one gene.

The DNA may be obtained from any cell source. Non-limiting examples of cell sources available in clinical practice include without limitation blood cells, buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates (e.g., pus) at a site of infection or inflammation. DNA is extracted from the cell source or body fluid using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs).

A “sample” as used herein refers to a biological sample, such as, for example, tissue (or cells) or fluid isolated from an individual or from in vitro cell culture constituents, as well as samples obtained from the environment or laboratory procedures.

Determination of the sequence of the extracted DNA at polymorphic positions is achieved by any means known in the art, including but not limited to direct sequencing, hybridization with allele-specific oligonucleotides, allele-specific PCR, ligase-PCR, HOT cleavage, denaturing gradient gel electrophoresis (DGGE), and single-stranded conformational polymorphism (SSCP). Direct sequencing may be accomplished by any method, including without limitation chemical sequencing, using the Maxam-Gilbert method; by enzymatic sequencing, using the Sanger method; mass spectrometry sequencing; and sequencing using a chip-based technology. See, e.g., Little et al., Genet. Anal. 6:151, 1996. Preferably, DNA from a subject is first subjected to amplification by polymerase chain reaction (PCR) using specific amplification primers.

“Amplification” of DNA as used herein denotes the use of polymerase chain reaction (PCR) to increase the concentration of a particular DNA sequence within a mixture of DNA sequences. For a description of PCR see Saiki et al., Science, 239:487, 1988.

“Chemical sequencing” of DNA denotes methods such as that of Maxam and Gilbert (Maxam-Gilbert sequencing, Maxam and Gilbert, Proc. Natl. Acad. Sci. USA, 74:560, 1977), in which DNA is randomly cleaved using individual base-specific reactions.

“Enzymatic sequencing” of DNA denotes methods such as that of Sanger (Sanger et al., 1977, Proc. Natl. Acad. Sci. USA, 74:5463, 1977), in which a single-stranded DNA is copied and randomly terminated using DNA polymerase, including variations thereof well-known in the art.

The term “single-strand conformational polymorphism analysis” (SSCP) refers to a method for detecting sequence differences between two DNAs, comprising hybridization of the two species with subsequent mismatch detection by gel electrophoresis. (Ravnik-Glavac et al., Hum. Mol. Genet., 3:801, 1994.)

“HOT cleavage” is defined herein as a method for detecting sequence differences between two DNAs, comprising hybridization of the two species with subsequent mismatch detection by chemical cleavage (Cotton, et al., Proc. Natl. Acad. Sci., USA, 85:4397, 1988).

“Denaturing gradient gel electrophoresis” (DDGE) refers to a method for resolving two DNA fragments of identical length on the basis of sequence differences as small as a single base pair change, using electrophoresis through a gel containing varying concentrations of denaturant (Guldberg et al., Nuc. Acids Res., 22:880,;1994.)

As used herein, “sequence-specific oligonucleotides” refers to related sets of oligonucleotides that can be used to detect allelic variations or mutations in the prostanoid receptor genes.

A “probe” refers to a nucleic acid or oligonucleotide that forms a hybrid structure with a sequence in a target region due to complementarity of at least one sequence in the probe with a sequence in the target protein.

In an alternate embodiment, biopsy tissue is obtained from a subject. Antibodies that are capable of distinguishing between different polymorphic forms of prostaglanlin receptor are then contacted with samples of the tissue to determine the presence or absence of a polymorphic form specified by the antibody. The antibodies may be polyclonal or monoclonal, preferably monoclonal. Measurement of specific antibody binding to cells may be accomplished by any known method, e.g., quantitative flow cytometry, or enzyme-linked or fluorescence-linked immunoassay. The presence or absence of a particular polymorphism or polymorphic pattern, and its allelic distribution (i.e., homozygosity vs. heterozygosity) is determined by comparing the values obtained from a patient with norms established from populations of patients having known polymorphic patterns.

In another alternate embodiment, RNA is isolated from biopsy tissue using standard methods well known to those of ordinary skill in the art such as guanidium thiocyanate-phenol-chloroform extraction (Chomocyznski et al., Anal. Biochem., 162:156, 1987). The isolated RNA is then subjected to coupled reverse transcription and amplification by polymerase chain reaction (RT-PCR), using specific oligonucleotide primers that are specific for a selected polymorphism. Conditions for primer annealing are chosen to ensure specific reverse transcription and amplification; thus, the appearance of an amplification product is diagnostic of the presence of a particular polymorphism. In another embodiment, RNA is reverse-transcribed and amplified, after which the amplified sequences are identified by, e.g., direct sequencing. In still another embodiment, cDNA obtained from the RNA can be cloned and sequenced to identify a polymorphism.

Establishing Reference Polymorphism Patterns

In practicing the present invention, the distribution of polymorphic patterns in a large number of individuals exhibiting particular prostanoid responsivity status is determined by any of the methods described above, and compared with the distribution of polymorphic patterns in patients that have been matched for age, ethnic origin, and/or any other statistically or medically relevant parameters, who exhibit quantitatively or qualitatively different prostanoid responsivity status. Correlations are achieved using any method known in the art, including nominal logistic regression or standard least squares regression analysis. In this manner, it is possible to establish statistically significant correlations between particular polymorphic patterns and particular prostanoid responsivity statuses. It is further possible to establish statistically significant correlations between particular polymorphic patterns and changes in prostanoid responsivity status such as, would result, e.g., from particular treatment regimens. Thus, it is possible to correlate polymorphic patterns with responsivity to particular treatments.

A statistically significant correlation preferably has a “p” value of less than or equal to 0.05. Any standard statistical method can be used to calculate these values, such as the normal Student's T Test, or Fischer's Exact Test.

The identity and number of polymorphisms to be included in a reference pattern depends not only on the prevalence of a polymorphism and its predictive value for the particular use, but also on the value of the use and its requirement for accuracy of prediction. The greater the predictive value of a polymorphism, the lower the need for inclusion of more than one polymorphism in the reference pattern. However, if a polymorphism is very rare, then its absence from an individual's pattern might provide no indication as to whether the individual has a particular status. Under these circumstances, it might be advisable to select instead two or more polymorphisms which are more prevalent. Even if none of them has a high predictive value on its own, the presence of both (or all three) of them might be sufficiently predictive for the particular purpose.

If for example the use for a reference pattern is prediction of response to a drug, and among the afflicted population only a 30% response to the drug is observed, the reference pattern need only permit selection of a population that improves the response rate by 10% to provide a significant improvement in the state of the art. On the other hand, if the use for the reference pattern is selection of subjects for a particular clinical study, the pattern should be as selective as possible and should therefore include a plurality of polymorphisms that together provide a high predictive accuracy for the intended response.

In establishing reference polymorphism patterns, it is desirable to use a defined population. For example, tissue libraries collected and maintained by state or national departments of health can provide a valuable resource, since genotypes determined from these samples can be matched with medical history, and particularly prostanoid responsivity status, of the individual. Such tissue libraries are found, for example, in Sweden, Iceland, Norway, and Finland. As can be readily understood by one of ordinary skill in the art, specific polymorphisms may be associated with a closely linked population. However, other polymorphisms in the same gene may correlate with prostanoid responsivity status of other genetically related populations. Thus, in addition to the specific polymorphisms provided in the instant application, the invention identifies genes in which any polymorphisms can be used to establish reference and test polymorphism patterns for evaluating prostanoid responsivity status of individuals in the population.

In a specific embodiment, for example, DNA samples can be obtained form a well defined population, such as Caucasian males born in Uppsala, Sweden between 1920 and 1924. In a specific embodiment, such individuals are selected for the test population based on their medical history, i.e., they were either (i) healthy, with no signs of hypertension (or any cardiovascular condition) or glaucoma; or (ii) had suffered from hypertension or glaucoma.

In a specific embodiment, DNA sequence analysis can be carried out by: (i) amplifying short fragments of each of the genes using polymerase chain reaction (PCR) and (ii) sequencing the amplified fragments. The sequences obtained from each individual can then be compared with the first known sequences, e.g., to identify polymorphic positions.

A prostaglandin receptor gene or cDNA corresponding to a particular sequence is understood to include alterations in the particular sequence that do not change the inherent properties of the sequence. It will be understood that additional nucleotides may be added to the 5′ or 3′ termini of the genes shown in FIGS. 1 and 2 as part of routine recombinant DNA manipulations. Furthermore, sequence-conservative DNA substitutions, i.e., changes in the sequence of the protein-coding region that do not change the encoded amino acid sequence, may also be accommodated.

An “immunogenic component” as used herein refers to a protein, peptide, or chemical entity which can elicit the production of specific antibodies, i.e., antibodies which bind with high affinity to the specific protein, peptide, or chemical entity.

“Therapeutic regimen”, as used herein, refers without limitation to methods for the elimination or amelioration of symptoms and events associated with IOP or glaucoma. Such methods include, without limitation, alteration in diet, lifestyle, and exercise regimen; invasive and noninvasive surgical techniques such as laser surgery and cryosurgery; and pharmaceutical interventions, such as administration of prostaglandin agonists and antagonists. Intervention with pharmaceutical agents not yet known whose activity correlates with particular allelic patterns associated with different patterns of responsiveness to prostanoids is also encompassed by “therapeutic regimen”.

“Most positive treatment outcome” as used herein refers the result of the administration or application of a treatment regimen which most effectively ameliorates, eliminates, or prevents occurrence of IOP or glaucoma, with the fewest and least severe side effects.

The present inventors have surprisingly and unexpectedly discovered a number of genetic polymorphisms within each of the human genes encoding the FP prostaglandin receptor and the EP-1 prostaglandin receptor. The isolation and sequencing of the FP and EP-1 genes is described in detail in Example 1 below. The sequence of the FP receptor, including the disclosed polymorphisms, is depicted in FIG. 1, and the sequence of the EP-1 receptor, including the disclosed polymorphisms, is depicted in FIG. 2. The present invention thus provides isolated nucleic acids comprising polymorphic FP and EP-1 receptor sequences; polypeptides encoded by these amino acids; antibodies that recognize and discriminate FP and EP-1 variants; and methods for using the isolated nucleic acids, polypeptides, and/or antibodies for detecting these polymorphisms in individuals within the general population.

For isolation and identification of polymorphic FP receptor and EP-1 receptor DNA, polymerase chain reaction (PCR) was used to amplify PP and EP-1 sequences from human genomic DNA. The amplified products were sequenced, and the sequences were compared with FP and EP-1 sequences previously deposited in the European Molecular Biology Laboratory (EMBL) database with accession numbers L24470 (FP) and L22647 (EP-1).

Table 1 shows allelic polymorphisms in the FP and EP-1 genes according to the present invention. The designation of nucleotide number one is assigned to the first nucleotide in the start-(ATG) codon of the translated protein. For introns, the designation of nucleotide number one is assigned to the first nucleotide in the intron after the exon-intron splice site. For amino acid positions, the first amino acid to be translated is designated amino acid number one.

TABLE 1
Location of Polymorphisms in the Genes Encoding the FP-receptor and the EP1-receptor

| | | | | Resulting | | |
|---|---|---|---|---|---|---|
| | Nucleotide | Exon (e)/ | Nucleotide | amino acid | Name of | |
| Gene | position | Intron (i) | variation | variation | fragment | Frequency |
| FP receptor | 63 | e2 | C to T | None | FPF1, FPF1 | TT: 76% CT: 24% |
| FP receptor | 213 | e2 | C to T | None | FPF1, FPF1 | TT: 100% |
| FP receptor | 465 | e2 | G to A | Met to Ile | FPF2, FPF2 | GG: 96% GA: 4% |
| FP receptor | 573 | e2 | A to G | None | FPF3, FPR3 | AA: 96% AG: 4% |
| FP receptor | 292 | i2 | 2 bp insertion | None | FPFi2 | Unknown |
| FP receptor | 1012 | e3 | A to G | Ile to Val | FPF4, FPR4 | AA: 92% AG: 8% |
| EP1 receptor | 211 | e2 | A to G | Thr to Ala | EP1F1, EP1R1 | GG: 100% |
| EP1 receptor | 264 | e2 | C to T | None | EP1F1, EP1R1 | CC: 96% CT: 4% |
| EP1 receptor | 689 | e2 | A to T | His to Leu | EP1R3, | TT: 100% |
| | | | | | EP1F2.5 | |
| EP1 receptor | 690 | e2 | T to A | His to Leu | EP1R3, | AA: 100% |
| | | | | | EP1F2.5 | |
| EP1 receptor | 767 | e2 | A to G | His to Arg | EP1R3, | AA: 96% AG:4% |
| | | | | | EP1F2.5 | |
| EP1 receptor | 816 | e2 | C to T | None | EP1R3, | CC: 96% CT: 4% |
| | | | | | EP1F2.5 | |
| EP1 receptor | 93 | i2 | C to T | None | EPF1, EPF1 | CC: 92% CT: 8% |
| EP1 receptor | 999 | e3 | G to A | None | EP1F4, EP1R4, | GG: 64% GA: 24% |
| | | | | | EP1R4K | AA: 12% |

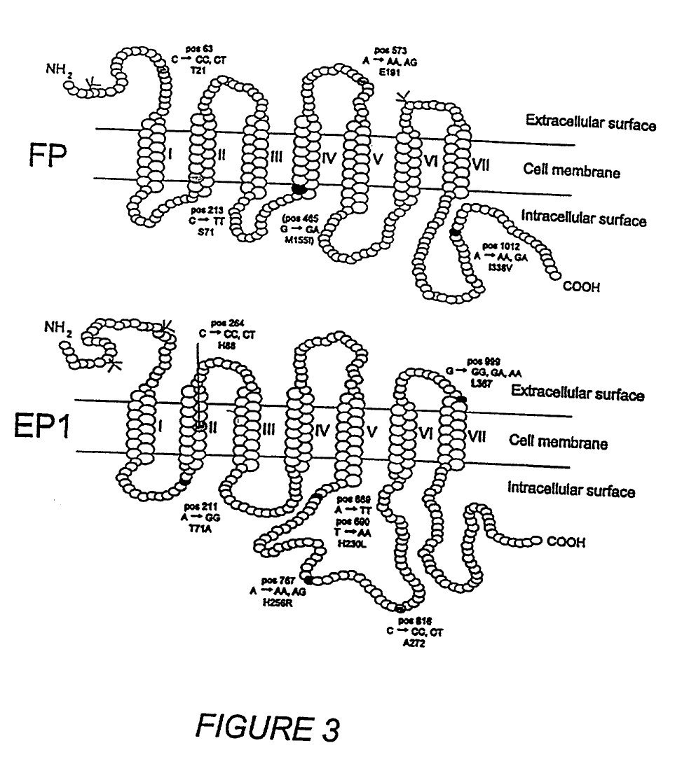

In the FP receptor, a polymorphism at nucleotide 465 results in a Met->Ile substitution at amino acid position 155, which is within the third intracellular loop of the receptor that is believed to be involved in binding to G proteins (FIG. 3). A polymorphism at nucleotide 1012 results in an Ile->Val substitution at amino acid 338, which is predicted to lie in the intracellular carboxyterminal tail of the protein (FIG. 3).

In the EP-1 receptor, a polymorphism at nucleotide 211 results in a Thr->Ala substitution at amino acid 71. Three polymorphisms result in amino acid substitutions within the third intracellular loop (FIG. 3). Variation at nucleotides 689 and 690 results in a His->Leu substitution at amino acid 230, and variation at nucleotide 767 results in a His->Arg substitution at amino acid 256. Other polymorphisms illustrated in Table 3 above are not predicted to result in amino acid substitutions in the respective receptors.

Polymorphisms that do not alter amino acid sequence could still have an important biological function that can affect responsiveness to prostanoid treatment. Such polymorphisms could affect regulation of transcription or translation, e.g., mRNA stability, splicing, transcription rate, translation rate, translation fidelity, etc.

The elucidation of significant genetic polymorphism in the FP and EP-1 receptor genes is likely to reflect a wide variation in the responses of an individual to different prostanoids, in terms of dose-and time-dependence of response; maximal level of response; specificity; and toxic and other side-effects. The present invention takes advantage of these genetic polymorphisms to predict how an individual might respond to different therapeutic regimens involving prostanoid administration. These embodiments of the invention are described in more detail below.

The present invention encompasses an isolated nucleic acid comprising the sequences depicted in FIG. 1. In particular, the invention encompasses FP prostaglandin receptor-encoding sequences comprising the DNA sequence defined by the nucleotides located at positions 63, 213, 465, 573, and 1012 of FIG. 1, which are listed in Table 1 above. Thus, the invention provides FP-encoding nucleic acids containing one or more of the listed polymorphisms at positions 63, 213, 465, 573, and 1012. Also included are sequences comprising the DNA sequence defined by the nucleotides located at position 212 of the second intron of FP.

The invention also provides an isolated nucleic acid derived from the sequence of FIG. 1 and encoding a polypeptide possessing the ligand-binding and activation properties of human FP prostaglandin receptor, in particular high affinity binding of [³H] PGF_{2d} that is displaceable by fluprostenal, as well as sequence-conservative and function-conservative variants thereof.

In another aspect, the invention encompasses an isolated nucleic acid comprising the sequences depicted in FIG. 2. In particular, the invention encompasses EP-1 prostaglandin receptor-encoding sequences comprising the DNA sequence defined by the nucleotides located at positions 211, 689, 690, 767, and 999 of FIG. 2, which are listed in Table 1 above. Thus, the invention provides EP-1-encoding nucleic acids containing one or more of the listed polymorphisms at positions 211, 689, 690, 767, and 999. Also included are sequences comprising the DNA sequence defined by the nucleotides located at position 93 of the second intron of EP-1.

The invention also provides an isolated nucleic acid derived from the sequence of FIG. 2 and encoding a polypeptide possessing the ligand-binding and activation properties of human EP-1 prostaglandin receptor, in particular high affinity binding of [³H] PGE₂ that is displaceable by sulprostone, iloprost, and 17-phenyl-trinor PGE₂, as well as sequence-conservative and function-conservative variants thereof.

Also encompassed by the invention is any nucleic acid hybridizable to, or derived from, the nucleic acids of FIGS. 1 and 2. In one embodiment, the invention relates to nucleotide probes capable of hybridizing with the nucleic acids of FIG. 1 or FIG. 2, or with its complementary sequences, as well as with the messenger RNA encoding FP or EP-1 prostaglandin receptors under the hybridization conditions defined below.

Prehybridization treatment of the support (nitrocellulose filter or nylon membrane), to which is bound the nucleic acid capable of hybridizing with that of FIG. 1 or FIG. 2, at 65° C. for 6 hours with a solution having the following composition: 4× SSC, 10× Denhardt (1× Denhardt is 1% Ficoll, 1% polyvinylpyrrolidone, 1% BSA (bovine serum albumin); 1× SSC consists of 0.15M of NaCl and 0.015M of sodium citrate, pH 7);

Replacement of the pre-hybridization solution in contact with the support by a buffer solution having the following composition: 4× SSC, 1× Denhardt, 25 mM NaPO4, pH 7,2 mM EDTA, 0.5% SDS, 100 mu g/ml of sonicated salmon sperm DNA containing a nucleic acid derived from the sequence of FIG. 1 or FIG. 2 as probe, in particular as radioactive probe, and previously denatured by a treatment at 100° C. for 3 minutes;

Incubation for 12 hours at 65° C.;

Successive washings with the following solutions: (i) four washings with 2× SSC, 1× Denhardt, 0.5% SDS for 45 minutes at 65° C.; (ii) two washings with 0.2× SSC, 0.1× SSC for 45 minutes at 65° C.; and (iii) 0.1× SSC, 0.1% SDS for 45 minutes at 65° C.

The invention also encompasses any nucleic acid exhibiting the property of hybridizing specifically with the nucleic acid of FIG. 1 or FIG. 2 under non-stringent conditions, which includes hybridization in the solution described above, but at 40° C., and includes successive washings in 2× SSC at 45° C. for 15 minutes.

It will be understood that the stringent or non-stringent conditions of hybridization defined above constitute preferred conditions for the hybridization, but are in no way limiting and may be modified without in any way affecting the properties of recognition and hybridization of the probes and nucleic acids mentioned above.

The salt conditions and temperature during the hybridization and the washing of the membranes can be modified in the sense of a greater or lesser stringency without the detection of the hybridization being affected. For example, it is possible to add formamide in order to lower the temperature during hybridization.

DNA, Vectors, and Host Cells

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning, the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Insertion of nucleic acids (typically DNAs) encoding the polypeptides of the invention into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, any site desired may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., Science, 239:48, 1988. The cleaved vector and the DNA fragments may also be modified if required by homopolymeric tailing.

In certain embodiments, the invention encompasses isolated nucleic acid fragments comprising all or part of the individual nucleic acid sequences disclosed herein. The fragments are at least about 8 nucleotides in length, preferably at least about 12 nucleotides in length, and most preferably at least about 15-20 nucleotides in length.

The nucleic acids may be isolated directly from cells. Alternatively, the polymerase chain reaction (PCR) method can be used to produce the nucleic acids of the invention, using either chemically synthesized strands or genomic material as templates. Primers used for PCR can be synthesized using the sequence information provided herein and can further be designed to introduce appropriate new restriction sites, if desirable, to facilitate incorporation into a given vector for recombinant expression.

The nucleic acids of the present invention may be flanked by natural FP or EP-1 regulatory sequences, or may be associated with heterologous sequences, including promoters, enhancers, response elements, signal sequences, polyadenylation sequences, introns, 5′- and 3′-noncoding regions, and the like. The nucleic acids may also be modified by many means known in the art. Non-limiting examples of such modifications include methylation, “caps”, substitution of one or more of the naturally occurring nucleotides with an analog, internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs are also included. The nucleic acid may be derivatized by formation of a methyl or ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

The invention also provides nucleic acid vectors comprising the disclosed FP and EP-1-derived sequences or derivatives or fragments thereof. A large number of vectors, including plasmid and fungal vectors, have been described for replication and/or expression in a variety of eukaryotic and prokaryotic hosts, and may be used for gene therapy as well as for simple cloning or protein expression.

The encoded FP and EP-1 polypeptides may be expressed by using many known vectors, such as pUC plasmids, pET plasmids (Novagen, Inc., Madison, Wis.), or pRSET or pREP (Invitrogen, San Diego, Calif.), and many appropriate host cells, using methods disclosed or cited herein or otherwise known to those skilled in the relevant art. The particular choice of vector/host is not critical to the practice of the invention.

Recombinant cloning vectors will often include one or more replication systems for cloning or expression, one or more markers for selection in the host, e.g. antibiotic resistance, and one or more expression cassettes. The inserted FP or EP-1 coding sequences may be synthesized by standard methods, isolated from natural sources, or prepared as hybrids, etc. Ligation of the FP or EP-1 coding sequences to transciptional regulatory elements and/or to other amino acid coding sequences may be achieved by known methods. Suitable host cells may be transformed/transfected/infected as appropriate by any suitable method including electroporation, CaCl₂ mediated DNA uptake, fungal infection, microinjection, microprojectile, or other established methods.

Appropriate host cells included bacteria, archebacteria, fungi, especially yeast, and plant and animal cells, especially mammalian cells. Of particular interest are E. coli, B. Subtilis, S. aureus, Saccharomyces cerevisiae, Schizosaccharomyces pombi, SF9 cells, C129 cells, 293 cells, Neurospora, and CHO cells, COS cells, HeLa cells, and immortalized mammalian myeloid and lymphoid cell lines. Preferred replication systems include M13, ColE1, SV40, baculovirus, lambda, adenovirus, and the like. A large number of transcription initiation and termination regulatory regions have been isolated and shown to be effective in the transcription and translation of heterologous proteins in the various hosts. Examples of these regions, methods of isolation, manner of manipulation, etc. are known in the art. Under appropriate expression conditions, host cells can be used as a source of recombinantly produced FP or EP-1-derived peptides and polypeptides.

Advantageously, vectors may also include a transcription regulatory element (i.e., a promoter) operably linked to the FP or EP-1 receptor portion. The promoter may optionally contain operator portions and/or ribosome binding sites. Non-limiting examples of bacterial promoters compatible with E. coli include: β-lactamase (penicillinase) promoter, lactose promoter, tryptophan (trp) promoter, araBAD (arabinose) operon promoter, lambda-derived P₁ promoter and N gene ribosome binding site; and the hybrid tac promoter derived from sequences of the trp and lac UV5 promoters. Non-limiting examples of yeast promoters include 3-phosphoglycerate kinase promoter, glyceraldehyde-3-phosphate dehydrogenase (GAPDH) promoter, galactokinase (GAL1) promoter, galactoepimerase promoter, and alcohol dehydrogenase (ADH) promoter. Suitable promoters for mammalian cells include without limitation viral promoters such as that from Simian Virus 40 (SV40), Rous sarcoma virus (RSV), adenovirus (ADV), and bovine papilloma virus (BPV). Mammalian cells may also require terminator sequences and polyA addition sequences and enhancer sequences which increase expression may also be included; sequences which cause amplification of the gene may also be desirable. Furthermore, sequences that facilitate secretion of the recombinant product from cells, including, but not limited to, bacteria, yeast, and animal cells, such as secretory signal sequences and/or prohormone pro region sequences, may also be included. These sequences are well described in the art.

Nucleic acids encoding wild-type or variant FP or EP-1-derived polypeptides may also be introduced into cells by recombination events. For example, such a sequence can be introduced into a cell, and thereby effect homologous recombination at the site of an endogenous gene or a sequence with substantial identity to the gene. Other recombination-based methods such as nonhomologous recombinations or deletion of endogenous genes by homologous recombination may also be used.

The nucleic acids of the present invention find use as probes for the detection of genetic polymorphisms and as templates for the recombinant production of normal or variant FP or EP-1 prostaglandin receptor-derived peptides or polypeptides.

Prostaglandin Receptor Polypeptides

The present invention encompasses isolated peptides and polypeptides encoded by all or a portion of prostaglandin receptors comprising polymorphic positions disclosed above and in particular, polypeptides which comprise part or all of the amino sequences shown in FIGS. 1 and 2, as well as all of the polypeptides possessing ligand-binding and/or G-protein activation activity of the FP or EP-1 prostaglandin receptors type from which they are derived and which are encoded in the above-mentioned DNA fragments derived from the nucleic acids of FIG. 1 or FIG. 2. In one preferred embodiment, the peptides and polypeptides are useful screening targets to identify drugs. In another preferred embodiment, the peptides and polypeptides are capable of eliciting antibodies in a suitable host animal that react specifically with a polypeptide comprising the polymorphic position and distinguish it from other polypeptides having a different amino acid sequence at that position.

Methods for identifying and selecting those polypeptides with shorter sequences which are encompassed by the invention are well-known in the art. For example, enzymatic or chemical proteolysis of FP or EP-1 receptors can be performed, with subsequent isolation of fragments and assay for FP or EP-1 prostaglandin receptor activity. Alternatively, FP or EP-1 polypeptides can be expressed recombinantly, and the expressed polypeptides can be assayed for FP or EP-1 prostaglandin receptor activity (Lanzillo, et al., J. Biol. Chem. 260(28):14938-14944, 1985; Welsch, et al., J. Cardiovasc. Pharmacol. 14(supp. 4):S26-S31, 1989).

Polypeptides according to the invention are preferably at least five or more residues in length, preferably at least fifteen residues. Methods for obtaining these polypeptides are described below. Many conventional techniques in protein biochemistry and immunology are used. Such techniques are well known and are explained in Immunochemical Methods in Cell and Molecular Biology, 1987 (Mayer and Waler, eds; Academic Press, London); Scopes, 1987, Protein Purification: Principles and Practice, Second Edition (Springer-Verlag, N.Y.) and Handbook of Experimental Immunology, 1986, Volumes I-IV (Weir and Blackwell eds.).

Nucleic acids comprising protein-coding sequences can be used to direct the recombinant expression of prostaglandin receptor-derived polypeptides in intact cells or in cell-free translation systems. The known genetic code, tailored if desired for more efficient expression in a given host organism, can be used to synthesize oligonucleotides encoding the desired amino acid sequences. The phosphoramidite solid support method Metteucci et al., J. Am. Chem. Soc., 103:3185, 1981; Yoo et al., J. Biol. Chem., 764:17078, 1989; or other well-known methods can be used for such synthesis. The polypeptides may be isolated from human cells into which an appropriate protein-coding sequence has been introduced and expressed. Furthermore, the polypeptides may be part of recombinant fusion proteins.

Peptides and polypeptides may be chemically synthesized by commercially available automated procedures, including, without limitation, exclusive solid phase synthesis, partial solid phase methods, fragment condensation or classical solution synthesis. The polypeptides are preferably prepared by solid phase peptide synthesis as described by Merrifield, J. Am. Chem. Soc., 85:2149, 1963.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. In a specific embodiment, a prostaglandin receptor can be purified by affinity chromatography with a prostanoid, e.g., prostaglandin. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against prostaglandin receptor or against peptides derived therefrom, can be used as purification reagents. Other purification methods are possible.

The present invention also encompasses derivatives and homologues of the polypeptides. For some purposes, nucleic acid sequences encoding the peptides may be altered by substitutions, additions, or deletions that provide for functionally equivalent molecules, i.e., function-conservative variants. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

The isolated polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds.

Polymorphism-Specific Antibodies

The present invention also encompasses antibodies that specifically recognize the polymorphic positions of the invention and distinguish a peptide or polypeptide containing a particular polymorphism from one that contains a different sequence at that position. Such polymorphic position-specific antibodies according to the present invention include polyclonal and monoclonal antibodies. The antibodies may be elicited in an animal host by immunization with prostaglandin receptor-derived immunogenic components or may be formed by in vitro immunization of immune cells. The immunogenic components used to elicit the antibodies may be isolated from human cells or produced in recombinant systems. The antibodies may also be produced in recombinant systems programmed with appropriate antibody-encoding DNA. Alternatively, the antibodies may be constructed by biochemical reconstitution of purified heavy and light chains. The antibodies include hybrid antibodies (i.e., containing two sets of heavy chain/light chain combinations, each of which recognizes a different antigen), chimeric antibodies (i.e., in which either the heavy chains, light chains, or both, are fusion proteins), and univalent antibodies (i.e., comprised of a heavy chain/light chain complex bound to the constant region of a second heavy chain). Also included are Fab fragments, including Fab′ and F(ab)₂ fragments of antibodies. Methods for the production of all of the above types of antibodies and derivatives are well-known in the art and are discussed in more detail below. For example, techniques for producing and processing polyclonal antisera are disclosed in Mayer and Walker, 1987, Immunochemical Methods in Cell and Molecular Biology, (Academic Press, London). The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. See, e.g., Schreier et al., 1980, Hybridoma Techniques; U.S. Pat. Nos. 4,341,761; 4,399,121; 4,427,783; 4,444,887; 4,466,917; 4,472,500; 4,491,632; and 4,493,890. Panels of monoclonal antibodies produced against ACE, AGT, or AT1 -derived epitopes can be screened for various properties; i.e. for isotype, epitope affinity, etc.

Many references are available for guidance in applying any of the above techniques: Kohler et al., 1980, Hybridoma Techniques, Cold Spring Harbor Laboratory, New York; Tijssen, 1985, Practice and Theory of Enzyme Immunoassays, Elsevier, Amsterdam; Campbell, 1984, Monoclonal Antibody Technology, Elsevier, Amsterdam; Hurrell, 1982, Monoclonal Hybridoma Antibodies: Techniques and Applications, CRC Press, Boca Raton, Fla. Monoclonal antibodies can also be produced using well known phage library systems.

The antibodies of this invention can be purified by standard methods, including but not limited to preparative disc-gel electrophoresis, isoelectric focusing, HPLC, reversed-phase HPLC, gel filtration, ion exchange and partition chromatography, and countercurrent distribution. Purification methods for antibodies are disclosed, e.g., in The Art of Antibody Purification, 1989, Amicon Division, W. R. Grace & Co. General protein purification methods are described in Protein Purification: Principles and Practice, R. K. Scopes, Ed, 1987, Springer-Verlag, New York, N.Y.

Methods for determining the immunogenic capability of the disclosed sequences and the characteristics of the resulting sequence-specific antibodies and immune cells are well-known in the art. For example, antibodies elicited in response to a peptide comprising a particular polymorphic sequence can be tested for their ability to specifically recognize that polymorphic sequence, i.e., to bind differentially to a peptide or polypeptide comprising the polymorphic sequence and thus distinguish it from a similar peptide or polypeptide containing a different sequence at the same position.

New Polymorphisms

The present invention also encompasses the identification and analysis of new alleles of prostaglandin receptor, such as FP and EP-1, genes that may be associated with variations in responses to prostanoid drugs. In this embodiment, genomic DNA may be amplified, or, alternatively, RNA encoding FP or EP-1 may be selectively reverse-transcribed and amplified as described above. The DNA product is then sequenced directly, and the sequence compared with the sequence of the known alleles of the gene of interest. Once a new allele has been identified, allele-specific DNA primers and/or allele-specific antibodies can be prepared by standard methods. These reagents can then be used for screening of individuals for FP or EP-1 alleles as described above.

In practicing the present invention, the distribution of FP and EP-1 allelic patterns in a large number of individuals exhibiting particular responses to prostanoids is determined by any of the methods described above, and compared with the distribution of FP and EP-1 allelic patterns in patients that have been matched for age and ethnic origin who exhibit different patterns of response. A statistical method such as a 2×3 Chi square test is then used to determine whether the allele frequencies in the groups are the same or different. In this manner, it is possible to establish statistically significant correlations between a given physiological status (including, e.g., efficacy of a particular treatment regimen) and previously known or novel allelic patterns of one or more FP and EP-1 genes. It is contemplated that correlations between particular FP and/or EP-1 allelic patterns and particular diseases will provide an important prognosticator of responsivity to particular treatment regimen.

Kits

The present invention further provides kits for the determination of the sequence at a polymorphic position or positions within the prostaglandin receptor gene in an individual. The kits comprise a means for determining the sequence at the polymorphic positions, and may optionally include data for analysis of polymorphic patterns. The means for sequence determination may comprise suitable nucleic acid-based and immunological reagents. Preferably, the kits also comprise suitable buffers, control reagents where appropriate, and directions for determining the sequence at a polymorphic position. The kits may also comprise data for correlation of particular polymorphic patterns with desirable treatment regimens or other indicators.

Nucleic-acid-based diagnostic methods and kits

The invention provides nucleic acid-based methods for detecting polymorphic patterns in a biological sample. The sequence at particular polymorphic positions in the genes is determined using any suitable means known in the art, including without limitation hybridization with polymorphism-specific probes and direct sequencing.

The present invention also provides kits suitable for nucleic acid-based diagnostic applications. In one embodiment, diagnostic kits include the following components:

(i) Probe DNA: The probe DNA may be pre-labelled; alternatively, the probe DNA may be unlabelled and the ingredients for labelling may be included in the kit in separate containers; and

(ii) Hybridization reagents: The kit may also contain other suitably packaged reagents and materials needed for the particular hybridization protocol, including solid-phase matrices, if applicable, and standards.

In another embodiment, diagnostic kits include:

(i) Sequence determination primers: Sequencing primers may be pre-labelled or may contain an affinity purification or attachment moiety; and

(ii) Sequence determination reagents: The kit may also contain other suitably packaged reagents and materials needed for the particular sequencing protocol. In one preferred embodiment, the kit comprises a panel of sequencing primers, whose sequences correspond to sequences adjacent to the polymorphic positions.

Antibody-based diagnostic methods and kits

The invention also provides antibody-based methods for detecting polymorphic patterns in a biological sample. The methods comprise the steps of: (i) contacting a sample with one or more antibody preparations, wherein each of the antibody preparations is specific for a particular polymorphic form of the prostaglandin receptor under conditions in which a stable antigen-antibody complex can form between the antibody and antigenic components in the sample; and (ii) detecting any antigen-antibody complex formed in step (i) using any suitable means known in the art, wherein the detection of a complex indicates the presence of the particular polymorphic form in the sample.

Typically, immunoassays use either a labelled antibody or a labelled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labelled immunoassays, such as ELISA assays.

The present invention also provides kits suitable for antibody-based diagnostic applications. Diagnostic kits typically include one or more of the following components:

(i) Polymorphism-specific antibodies: The antibodies may be pre-labelled; alternatively, the antibody may be unlabelled and the ingredients for labelling may be included in the kit in separate containers, or a secondary, labelled antibody is provided; and

(ii) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

Drug Targets and Screening Methods

According to the present invention, nucleotide sequences derived from the gene encoding a polymorphic form of a prostaglandin receptor, and peptide sequences derived from that polymorphic form of prostaglandin receptor, are useful targets to identify prostanoid drugs, i.e., compounds that are effective in treating one or more clinical symptoms of, for example, cardiovascular disease and glaucoma. Drug targets include without limitation (i) isolated nucleic acids derived from the gene encoding a prostaglandin receptor and (ii) isolated peptides and polypeptides derived from prostaglandin receptor polypeptides, each of which comprises one or more polymorphic positions.

In vitro screening methods

In one series of embodiments, an isolated nucleic acid comprising one or more polymorphic positions is tested in vitro for its ability to bind test compounds in a sequence-specific manner. The methods comprise:

(i) providing a first nucleic acid containing a particular sequence at a polymorphic position and a second nucleic acid whose sequence is identical to that of the first nucleic acid except for a different sequence at the same polymorphic position;

(ii) contacting the nucleic acids with a multiplicity of test compounds under conditions appropriate for binding; and

(iii) identifying those compounds that bind selectively to either the first or second nucleic acid sequence.

Selective binding as used herein refers to any measurable difference in any parameter of binding, such as, e.g., binding affinity, binding capacity, etc.

In another series of embodiments, an isolated peptide or polypeptide comprising one or more polymorphic positions is tested in vitro for its ability to bind test compounds in a sequence-specific manner. The screening methods involve:

(i) providing a first peptide or polypeptide containing a particular sequence at a polymorphic position and a second peptide or polypeptide whose sequence is identical to the first peptide or polypeptide except for a different sequence at the same polymorphic position;

(ii) contacting the polypeptides with a multiplicity of test compounds under conditions appropriate for binding; and

(iii) identifying those compounds that bind selectively to one of the nucleic acid sequences.

In preferred embodiments, high-throughput screening protocols are used to survey a large number of test compounds for their ability to bind the genes or peptides disclosed above in a sequence-specific manner.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., Tib Tech, 14:60, 1996).

In vivo screening methods

Intact cells or whole animals expressing polymorphic variants of a gene encoding prostaglandin receptor can be used in screening methods to identify candidate prostaglandin drugs.

In one series of embodiments, a permanent cell line is established from an individual exhibiting a particular polymorphic pattern. Alternatively, cells (including without limitation mammalian, insect, yeast, or bacterial cells) are programmed to express a gene comprising one or more polymorphic sequences by introduction of appropriate DNA Identification of candidate compounds can be achieved using any suitable assay, including without limitation (i) assays that measure selective binding of test compounds to particular polymorphic variants of prostaglandin receptor (ii) assays that measure the ability of a test compound to modify (i.e., inhibit or enhance) a measurable activity or function of the receptor and (iii) assays that measure the ability of a compound to modify (i.e., inhibit or enhance) the transcriptional activity of sequences derived from the promoter (i.e., regulatory) regions the prostaglandin receptor gene.

In another series of embodiments, transgenic animals are created in which (i) a human prostaglandin receptor having different sequences at particular polymorphic positions are stably inserted into the genome of the transgenic animal; and/or (ii) the endogenous prostaglandin receptor genes are inactivated and replaced with human prostaglandin receptor genes having different sequences at particular polymorphic positions. See, e.g., Coffman, Semin. Nephrol. 17:404, 1997; Esther et al., Lab. Invest. 74:953, 1996; Murakami et al., Blood Press. Suppl. 2:36, 1996. Such animals can be treated with candidate compounds and monitored for one or more clinical markers of prostanoid response status.

Furthermore, populations that are not amenable to an established treatment for a prostaglandin-associated disease or disorder can be selected for testing of alternative treatments. Moreover, treatments that are not as effective in the general population, but that are highly effective in the selected population, may be identified that otherwise would be overlooked. This is an especially powerful advantage of the present invention, since it eliminates some of the randomness associated with clinical trials.

High-Throughput Screen

Agents according to the invention may be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time. Such high-throughput screening methods are particularly preferred. The use of high-throughput screening assays to test for agents is greatly facilitated by the availability of large amounts of purified polypeptides, as provided by the invention.

Agents according to the invention are useful for preventing or treating one or more symptoms of IOP and/or glaucoma in susceptible mammals. Pharmaceutical formulations incorporate a prophylactically or therapeutically effective amount of one or more of the agents identified as described above. A prophylactically effective amount is an amount effective to prevent one or more symptoms of IOP and/or glaucoma, and will depend upon the symptoms, the agent, and the subject to whom the agent is administered. Similarly, a therapeutically effective amount is an amount effective to ameliorate one or more symptoms of IOP or glaucoma. These amounts can be determined experimentally by methods known in the art and as described above.

The agents of the invention can be administered to patients via oral and/or parental routes. Parental routes include, without limitation, intraocular, subcutaneous, intramuscular, intraperitoneal, intraduodenal, and intravenous administration. The prophylactically and/or therapeutically effective amounts can be administered in one administration or over repeated administrations. Therapeutic administration can be followed by prophylactic administration, and vice versa.

The following are intended as non-limiting examples of the invention.

### EXAMPLE 1

### Isolation and Determination of the Nucleic Acids Encoding Polymorphic Variants of the FP and EP1 Prostaglandin Receptor Genes

A. Isolation of genomic DNA

Genomic DNA was purified from the white blood cells obtained from 1.5 ml of a human blood sample. The isolated DNA was dissolved in 5 ml of TE-buffer (10 mM Tris-HCl, 1 mM EDTA, pH 8.0) for amplification by PCR.

B. PCR amplification

1) Strategy:

Genomic DNA was subjected to PCR using pairs of primers shown in Table 2 below:

TABLE 2
Primers used in PCR amplification of the regions of the FP-
and EP1-receptor genes displaying genetic variation

| | | Nucleotides | SEQ ID NO. |
|---|---|---|---|
| FP/3FT | 5′-TTGGCTTTTATCTCCACAACAA-3′ | 5′UTR1—1 | 5 |
| FP/4RB | 5′-B-GGGCACAGACCAGAAAACAC-3′ | 346-365 | 6 |
| FP/5FT | 5′-TGGAGCCATAGCAGTATTTGTATA-3′ | 252-275 | 7 |
| FP/6RB | 5′-B-GCCCCAGAAAAGAAAAAAGTAG-3′ | 607-628 | 8 |
| FP/10F | 5′-GCCCTTGGTGTTTCATTGTT-3′ | 634-653 | 9 |
| FP/11R | 5′-AGGATCTAAGATTTGATTCCATGTT-3′ | 879-903 | 10 |
| FP/13R | 5′-GGACAGCCTTTCGTAGAAGAATATA-3′ | 910-934 | 11 |
| FP/14R | 5′-GCACTCCACAGCATTGACTG-3′ | 957-976 | 12 |
| FP/15F | 5′-TAAAAGTCAGCAGCACAGACAAG-3′ | 695-718 | 13 |
| FP/16FT | 5′-GTCGAGGACCTGGTGTTTCTA-3′ | 543-564 | 14 |
| FP/17RB | 5′-B-AAATGGGCTCCAACAAATACAG-3′ | 773-795 | 15 |
| FP/18FT | 5′-CAACATTGGAATAAATGGAAATCA-3′ | 810-833 | 16 |
| FP/19RB | 5′-B-TAGCCCCACACAGATITACTGT-3′ | 1090-112 | 17 |
| FP/22FB | 5′-B-TTGGCTTTTATCTCCACAACA-3′ | 5′UTR1-21 | 18 |
| FP/23RT | 5′-GGGCACAGACCAGAAAACAC-3′ | 346-365 | 19 |
| FP/24FB | 5′-B-TGGAGCCATAGCAGTATTTGTATA-3′ | 252-275 | 20 |
| FP/25RT | 5′-GCCCCAGAAAAGAAAAAGTG-3′ | 607-628 | 21 |
| FP/26FB | 5′-B-CTGCCCATCCTTGGACATC-3′ | 505-523 | 22 |
| FP/27RT | 5′-AGTAGGGATCATTCTCAGCATTTA-3′ | intron2:81- | 23 |
| FP/28RB | 5′-B-CCAGAGAATGATTTCCATTTATTC-3′ | 104 | 24 |
| FP/33RT | 5′-CCCACACAGATTTACTGTCCTATT-3′ | 818-841 | 25 |
| FP/34F | 5′-AAATGCTGAGAATGATCCCTACTC-3′ | 1083-1107 | 26 |
| FP/35FB | 5′-B-TTGAAAAGGCTGCATCAACTAA-3′ | intron2:81- | 27 |
| EP1/5FB | 5′-B-CGCCTGACATGAGCCCTTGC-3′ | 104 | 28 |
| EP1/6RT | 5′-TGCAGCCGCCCAGGAAGTG-3′ | intron2/2:1- | 29 |
| EP1/10FB | 5′-B-GGCGAGGCGACCACATG-3′ | 22 | 30 |
| EP1/11RT | 5′-AGCAGCAGCGGGCACAG-3′ | 5′UTR1-12 | 31 |
| EP1/18FB | 5′-B-TTCATCGGCCTGGGTCC-3′ | 331-349 | 32 |
| EP1/19RT | 5′-CATTGGGCTCCAGCAGATG-3′ | 37-53 | 33 |
| EP1/21FT | 5′-CAGGGTGGGCTGGCTTAG-3′ | 363-380 | 34 |
| EP1/27FT | 5′-CTATAGCTCTTCTCCGGCTTCC-3′ | 565-582 | 35 |
| EP1/28RB | 5′-B-CAGGGTGGGCTGGCTTAGT-3′ | 922-939 | 36 |
| EP1/29FT | 5′-TTCATCGGCCTGGGTCC-3′ | 1231-1249 | 37 |
| EP1/30RB | 5′-B-TGCACGACACCACCATGATAC-3′ | intron2/2:1- | 38 |
| EP1/33FT | 5′-TCTGCCCTCCTCTCCTCTATC-3′ | 21 | 39 |
| EP1/34RB | 5′-B-GCCACAGCCCAGCAGCA-3′ | 1231-1249 | 40 |
| EP1/36FB | 5′-B-CTATAGCTCTTCTCCGGCTTCC-3′ | 565-582 | 41 |

The PCR primers shown in Table 2 were designated as follows:

FP: PCR primer for the amplification of the gene encoding the FP-receptor.

EP1: PCR primer for the amplification of the gene encoding the EP1-receptor.

F: Forward (defines the direction of the sequencing reaction).

R: Reverse (defines the direction of the sequencing reaction).

B: The PCR primer carries a biotin-molecule attached to the 5′-nucleotide of the primer.

T: Tail (the 29 bases defined as “Tail” below are added to the 5′-end of the PCR primer).

Tail: 5′-AGTCACGACGTTGTAAAACGACGGCCAGT-3′ (SEQ ID NO:42)

2) Reaction mixtures:

PCR reaction mixtures used in the amplification of FP and EP-1 nucleic acids were as follows:

PCR mix 1:

5 μl of 10×PCR buffer II (Perkin Elmer)

4 μl of 2.5 mM dNTP [dATP:dCTP:dGTP:dTTP=1:1:1:1] (Pharmacia Biotech)

3 μl of 25 mM MgCl₂ (Perkin Elmer)

2.5 μl DMSO (Pharmacia Biotech)

0.15 μl of AmpliTaq (5 U/μl) (Perkin Elmer)

1 μl of diluted genomic DNA solution

1 μl of each primer (10 pmol/μl)

33.35 μl ultrapure water

PCR mix 2:

5 μl of 10×PCR buffer II (Perkin Elmer)

4 μl of 2.5 mM dNTP [dATP:dCTP:dGTP:dTTP=4:4:1:3:4] (Pharmacia Biotech

3 μl of 25 mM MgCl₂ (Perkin Elmer)

2.5 μl DMSO (Pharmacia Biotech)

0.15 μl of AmpliTaq (5 U/μl) (Perkin Elmer)

1 μl of diluted genomic DNA solution

1 μl of each primer (10 pmol/μl)

33.35 ηl ultrapure water

PCR mix 3:

5 μl of 10×PCR buffer II (Perkin Elmer)

4 μl of 2.5 mM dNTP [dATP:dCTP:dTP:dTTP=2:2:1:1:2] (Pharmacia Biotech)

3 μl of 25 mM MgCl₂ (Perkin Elmer)

2.5 μl DMSO (Pharmacia Biotech)

0.15 μl of AmpliTaq (5 U/μl) (Perkin Elmer)

1 μl of diluted genomic DNA solution

1 μl of each primer (10 pmol/μl)

33.35 μl ultrapure water

3) Reaction conditions:

PCR reactions involved either nested or single PCR reactions For nested reactions, the protocol designated PCR1 below was used in the first reaction and that designated PCR2 was used in the subsequent reaction. For single reactions, the protocol designated PCR2 was used. For PCR2 reactions in nested PCR, 1 μl of the preceding PCR reaction was used as template.

PCR 1:

98° C. 3 min

3×(98° C. 15 sec, Tₐ° C. 30 sec, 72° 45 sec)

22×(95° C. 15 sec, Tₐ° C. 30 sec, 72° C. 45 sec)

72° C. 5 min

22° C. ∞

PCR 2:

98° C. 3 min

3×(98° C. 15 sec, Tₐ° C. 30 sec, 72° C. 45 sec)

40×(95° C. 15 sec, Tₐ° C. 30 sec, 72° C. 45 sec)

72° C. 5 min

22° C. ∞

4) Resulting fragments:

Table 3 below shows the pairs of primers that were employed in PCR reactions, the annealing temperature (Tₐ) used for each reaction, and the fragments that resulted.

TABLE 3

| Fragment | PCR primer 1 | PCR primer 2 | PCR conditions | T_{g} |
|---|---|---|---|---|
| FPF1 | FP/3FT | FP/4RB | 1 | 56 |
| FPR1 | FP/22FB | FP/23RT | 1 | 56 |
| FPF3 | FP/16FT | FP/17RB | 1 | 56 |
| FPR3 | FP/26FB | FP/27RT | 1 | 56 |
| FPF4 | FP/18FT | FP/19RB | 1 | 56 |
| FPR4 | FP/35FB | FP/33RT | 1 | 56 |
| FPFi2 PCR1 | FP/10F | FP/14R | 1 | 54 |
| FPFi2 PCR2 | FP/10F | FP/13R | 1 | 54 |
| FPFi2 PCR3 | FP/15F | FP/11R | 1 | 54 |
| FPFi2 PCR4 | FP/34F | FP/28RB | 1 | 52 |
| EP1F1 | EP1/33FT | EP1/34RB | 3 | 56 |
| EP1R1 PCR1 | EP1/5FB | EP1/11RT | 1 | 56 |
| EP1R1 PCR2 | EP1/10FB | EP1/6RT | 1 | 56 |
| EP1F2.5 | EP1/29FT | EP1/30RB | 3 | 62 |
| EP1R3 | EP1/18FB | EP1/19RT | 3 | 56 |
| EPAF4 | EP1/37FT | EP1/28RB | 3 | 56 |
| EP1R4 | EP1/36FB | EP1/21RT | 2 | 56 |
| EP1R4K PCR1 | EP1/27FT | EP1/28RB | 3 | 54 |
| EP1R4K PCR2 | EP1/38RT | EP1/39FB | 3 | 62 |

After each PCR reaction, 5 μl of the products were analyzed using agarose gel electrophoresis prior to nucleotide sequencing.

5) Sequencing:

Sequencing Using Solid-Phase Sequencing System on ALFexpress™

The sequence analysis of the PCR products from the exons and intron 2 of the EP1-receptor gene and the exons of the FP gene was performed by the solid-phase sequencing system method, commercially available as ALFexpress™ (Pharmacia Biotech, Uppsala, Sweden). The procedures were performed according to the instructions provided by Pharmacia Biotech.

Forty μl of the PCR-products were transferred to a 10-well plate and mixed with 80 μl BW-buffer (2 M NaCl, 10 mM Tris-HCl, 1 mM EDTA). The combs were inserted into the wells, dipped several times and left to stand at +4° C. over night (approximately 16-20 hr) to improve the capture of the PCR products on to the solid phase of the combs.

The DNA fragments bound to the combs were subjected to a denaturing step by incubating the combs in 0.1 M NaOH for 5 min. The combs were subsequently washed once in 10 mM Tris-HCl, pH 7.5.

The annealing mix, comprised of 104 μl of a Cy5labelled primer (1 pmol/μl) was added to a ten-well plate, and the comb carrying the denatured, washed PCR product was inserted. The annealing mix with the combs inserted was heated to 65° C. for 5 min., and then left at room temperature to cool.

20 μl of the sequence mix were dispensed into a 40-well plate, and the plate was kept on ice. The combs were inserted into the plate, and the plate was transferred to 42° C. for an incubation in 5 min. The plate was then transferred to ice. The sequence-mix contains 2 μl 10× annealing buffer, 1 μl extension buffer, 1 μl DMSO, 4 μl d/ddNTP mix, 11 μl water and 1 μl (2 units) T7 DNA polymerase diluted in enzyme dilution buffer. All components are commercially available as the Auto Load Kit (Pharmacia Biotech).

The ALFexpress™ gel (Pharmacia Biotech) was pre-warmed to 55° C., and the wells rinsed with the running buffer. The wells were filled with 100% STOP solution by the use of a syringe, and the combs were inserted and left to incubate for 10 min. The comb was removed and the run of the ALFexpress™ gel was commenced.

Sequencing Using Taq Dye Terminators on the ABI 377

The sequence analysis of the PCR products from intron 2 of the FP-receptor gene was performed by a cycle sequencing method, which is commercially available as the ABI PRISM Dye Terminator Cycle Sequencing Ready Reaction kit with AmpliTaq DNA Polymerase FS. The procedure were performed according to the instructions provided by Perkin Elmer. The primer used in the FP receptor intron 2 sequencing reaction had the following sequence:

FP/34F 5′-AAATGCTGAGAATGATCCCTACTC-3′

The PCR-product was purified with QiaQuick Spin columns from KEBO Lab, Sweden according to manual, and eluted in 30 μl ultrapure water.

1 μl purified DNA

4 μL Terminator Ready Reaction Mix

1.6 pmol primer

q.s. ultrapure water

10 μl final reaction volume

The cycling was performed on a Perkin Elmer 2400 or 9600 with the following cycle: 25×(96° C. 10 sec, 50° C. 5 sec, 60° C. 4 min.)−4° C.

The reactions were kept in a freezer unless the precipitation was done the same day. For precipitation, 2.0 μl 3M sodium acetate, pH 4.8, and 50 μl cold 95% ethanol were added to a 1.5 ml microcentrifuge tube. The reaction was transferred to the tube, vortexed, and allowed to precipitate for 10 min. Following centrifugation in a microcentrifuge at maximum speed for 15-30, the ethanol solution was carefully aspirated with a micropipette. The pellet was rinsed by adding 250 μl 70% ethanol and carefully aspirating all the alcohol solution with a micropipette. After drying the pellet for 30 minutes at room temperature, it was dissolved in 4.5 μl loading buffer included in the kit.

The ABI gel was pre-warmed to 55° C., and the wells rinsed with running buffer. 1.5 μl of the reaction product was applied to the wells of the gel and the run commenced.

6) Results:

The nucleotide sequences obtained using the above-described procedures are shown in FIGS. 1 and 2 (FP and EP-1 receptors, respectively) and in Table 1 above.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various patents, patent applications, publications, and procedures are cited throughout this application, the disclosures of which are incorporated herein by reference in their entireties.

1. A method for assessing prostanoid response status in an individual to be tested comprising
(a) comparing
(i) a test polymorphic pattern comprising at least one polymorphic position within a prostaglandin receptor gene of the individual, with
(ii) a reference polymorphic pattern derived from a population of individuals exhibiting a predetermined prostanoid response status; and
(b) concluding whether the individual possesses the prostanoid response status based on whether the test pattern matches the reference pattern.

2. The method according to claim 1, wherein the predetermined prostanoid response status is predisposition to glaucoma.

3. The method according to claim 1, wherein the predetermined prostanoid response status is predisposition to hypertension.

4. The method according to claim 1, wherein the predetermined prostanoid response status is responsivity to synthetic prostaglandin analogues.

5. The method according to claim 1, wherein the reference pattern comprises at least two polymorphisms.

6. The method according to claim 5, wherein the reference pattern comprises at least three polymorphisms.

7. A kit for assessing prostanoid response status comprising
(a) sequence determination oligonucleotides and
(b) sequence determination reagents,
wherein the primers are selected from the group consisting of primers that hybridize to or immediately adjacent to a polymorphic position in a human prostaglandin receptor gene.

8. The kit of claim 7, wherein the prostaglandin receptor is an FP prostaglandin receptor.

9. The kit of claim 8, wherein the polymorphism is of a nucleotide selected from the group consisting of nucleotide numbers 63, 213, 465, 573, and 1012 of a nucleic acid sequence as depicted in FIG. 1 (SEQ ID NO:1).

10. The kit of claim 7, wherein the prostaglandin receptor is an EP-1 prostaglandin receptor.

11. The kit of claim 7, wherein the polymorphism is of a nucleotide selected from the group consisting of nucleotide numbers 211, 264, 689, 690, 767, 816, and 999 of a nucleotide sequence as depicted in FIG. 2 (SEQ ID NO:3).

12. An isolated nucleic acid encoding a human FP prostaglandin receptor comprising the sequence depicted in FIG. 1 (SEQ ID NO:2), wherein said sequence comprises one or more residues selected from the group consisting of: a T residue at position 63; a T residue at position 213; an A residue at position 465; a G residue at position 573; and a G residue at position 1012.

13. A nucleic acid as defined in claim 12, wherein said nucleic acid is DNA.

14. A nucleic acid as defined in claim 12, wherein said nucleic acid is RNA.

15. A recombinant DNA vector comprising a nucleic acid as defined in claim 12 operably linked to a transcription regulatory element.

16. A cell comprising a DNA vector as defined in claim 15, wherein said cell is selected from the group consisting of bacterial, fungal, plant, insect, and mammalian cells.

17. A method for producing a polypeptide, said method comprising culturing a cell as defined in claim 16 under conditions that permit expression of one or more polypeptides encoded by said nucleic acid.

18. An isolated polypeptide having an amino acid sequence depicted in FIG. 1 (SEQ ID NO:2), wherein said polypeptide comprises any one or both of residues Ile₁₅₅ and Val₃₃₈.

19. A method of screening for a candidate compound that interacts with a human FP prostaglandin receptor comprising detecting binding of the polypeptide of claim 18 with the compound.

20. An isolated nucleic acid encoding a human EP-1 prostaglandin receptor comprising the sequence depicted in FIG. 2 (SEQ ID NO:4), wherein said sequence comprises one or more residues selected from the group consisting of: a G residue at position 211; a T residue at position 264; a T residue at position 689; an A residue at position 690; a G residue at position 767; a T residue at position 816; and an A residue at position 999.

21. A nucleic acid as defined in claim 20, wherein said nucleic acid is DNA.

22. A nucleic acid as defined in claim 20, wherein said nucleic acid is RNA.

23. A recombinant DNA vector comprising a nucleic acid as defined in claim 20 operably linked to a transcription regulatory element.

24. A cell comprising a DNA vector as defined in claim 23, wherein said cell is selected from the group consisting of bacterial, fungal, plant, insect, and mammalian cells.

25. A method for producing a polypeptide, said method comprising culturing a cell as defined in claim 24 under conditions that permit expression of one or more polypeptides encoded by said nucleic acid.

26. An isolated polypeptide having an amino acid sequence depicted in FIG. 2 (SEQ ID NO:4), wherein said polypeptide comprises any one or more of residues Ala₇₁; Leu₂₃₀; and Arg₂₅₆.

27. A method of screening for a candidate compound that interacts with a human EP-1 prostaglandin receptor comprising detecting binding of the polypeptide of claim 26 with the compound.
